(19) United States
(12) Patent Application Publication
(10) Pub. No.: US 2015/0031647 A1
(43) Pub. Date: Jan. 29, 2015

(54) TASTE MASKED DISPERSIBLE TABLETS

(71) Applicant: RANBAKY LABORATORIES LIMITED, New Delhi, Delhi (IN)

(72) Inventors: Vinod Kumar Arora, Gurgaon (IN); Jatin Khurana, North West Delhi (IN); Deepak Gaikwad, Mumbai (IN)

(73) Assignee: RANBAXY LABORATORIES LIMITED, New Delhi, Delhi (IN)

(21) Appl. No.: 14/379,861

(22) PCT Filed: Feb. 21, 2013
(86) PCT No.: PCT/IB2013/051432

§ 371 Date: Aug. 20, 2014

(30) Foreign Application Priority Data
Feb. 21, 2012 (IN) 496/DEL/2012

(51) Int. Cl. A61K 47/32; A61K 31/194; A61K 31/675

(52) U.S. Cl. CPC A61K 47/32; A61K 31/675; A61K 31/194
USPC 514/81

(57) ABSTRACT

The present invention relates to a taste-masked dispersible tablet comprising a drug, a cation exchange resin, and other pharmaceutically acceptable excipients, such that the said drug and the said cation exchange resin are present in an un-complexed form in the tablet. It further relates to a process for the preparation of the same.

### FIELD OF THE INVENTION

The present invention relates to a taste-masked dispersible tablet comprising a drug, a cation exchange resin, and other pharmaceutically acceptable excipients, such that the said drug and the said cation exchange resin are present in an un-complexed form in the tablet. It further relates to a process for the preparation of the same.

### BACKGROUND OF THE INVENTION

It is well known that oral administration constitutes a preferred route of administration for a majority of drugs. However, a drug having an inherently bitter taste constitutes a disadvantage with certain types of oral preparations, particularly when intended for use in the pediatric population. Unpleasant taste leads to poor adherence in children which in turn causes treatment failure. Therefore, taste-masking can prove to be an essential tool to improve patient compliance, especially of pediatric patients, by increasing the palatability of such bitter-tasting drugs. This assumes more importance for drugs that are used for a long-term cure and where treatment adherence is an important requisite.

Several methods are known in the prior art for taste-masking. To some extent, the bitter taste may be masked by the use of sweetening and/or flavoring agents. However, this method is not always satisfactory and an unpleasant taste may remain in the mouth. Additionally, there may be circumstances in which it is undesirable to use a sweetening agent and/or flavoring agent. Use of ion-exchange resins is an alternatively known method to mask the bitter taste in such cases.

U.S. Publication No. 2004/0067216 relates to a complex comprising a cation exchange resin and an HIV protease inhibitor and formulation of this drug-resin complex into a capsule dosage form.

U.S. Publication No. 2002/0032245 relates to a rapid-release resinate composition comprising an ion exchange resin loaded with an active substance, wherein the said active substance is anisotropically distributed throughout said ion exchange resin particle.

In the prior art, the drug-resin complexes are generally formed by mixing a drug with an aqueous suspension of a resin, after which the complex is filtered, washed, and dried. All these usual complexation steps are time-consuming and increase the cost of manufacturing. Further, this method is not suitable for drugs which are prone to hydrolysis and show poor stability in a solution form. The inventors have now developed a taste-masked dispersible tablet comprising a drug and a cation exchange resin which can be prepared by a simple procedure, does not involve the usual time consuming and costly steps of filtering, washing, drying, etc., and is suitable for drugs which are prone to hydrolysis. The inventors have also surprisingly found that the dispersible tablets wherein the drug-resin complex is formed in situ exhibit better stability than the dispersible tablets comprising the drug-resin complexes prepared by the usual complexation methods.

### SUMMARY OF THE INVENTION

In one general aspect, the present invention relates to a taste-masked dispersible tablet comprising a drug, a cation exchange resin, and other pharmaceutically acceptable excipients, such that the said drug and the said cation exchange resin are present in an un-complexed form in the tablet.

In one embodiment of the above aspect, the said drug and the said cation exchange resin form a complex within 30 seconds to 15 minutes of dispersing the taste-masked dispersible tablet in water.

In another embodiment of the above aspect, the cation exchange resin may be Amberlite® IRP64.

In the above aspect and embodiments, the other pharmaceutically acceptable excipients may be selected from diluents, binders, lubricants/glidants, disintegrants, flavoring agents, sweetening agents, coloring agents, or combinations thereof.

In another general aspect, the invention relates to a process for the preparation of a taste-masked dispersible tablet comprising a drug, a cation exchange resin, and other pharmaceutically acceptable excipients, such that the said drug and the said cation exchange resin are present in an un-complexed form in the tablet, wherein the process comprises the steps of direct compression, dry granulation, or wet granulation.

In an embodiment of the above aspect, the process comprises the steps of:
a) sifting all the ingredients, i.e., the drug, the cation exchange resin, and other pharmaceutically acceptable excipients separately through suitable sieves;
b) blending the sifted ingredients in a blender for a suitable time;
c) compressing the resultant blend into a dispersible tablet using appropriate tooling.

In another embodiment of the above aspect, the process comprises the steps of:
a) sifting the ingredients, i.e., the drug, the cation exchange resin, and other pharmaceutically acceptable excipients through suitable sieves;
b) blending the sifted ingredients in a blender for a suitable time;
c) granulating the resultant blend with a binder solution;
d) drying the granules;
e) mixing the granules with the extragranular excipient(s); and
f) compressing the resultant blend into a dispersible tablet using appropriate tooling.

In another general aspect, the present invention relates to a method of orally administering to a human a taste-masked dispersible tablet comprising a drug, a cation exchange resin, and other pharmaceutically acceptable excipients in water, such that the said drug and the said cation exchange resin are present in an un-complexed form in the tablet, wherein the method comprises dispersing said tablet in a sufficient quantity of water prior to administration.

In one embodiment of the above aspects, the said drug is tenofovir disoproxil fumarate.

### DETAILED DESCRIPTION OF THE INVENTION

The “cation exchange resin”, as recited herein, can be, for example, a copolymer of styrene or acrylic or methacrylic acid with a vinyl aromatic compound such as divinylbenzene, and the resin may derive its exchange activity from either weakly or strongly acidic groups such as carboxylic acid or sulphonic acid groups. Examples of suitable resins are those that are copolymers of styrene and divinylbenzene which are sulphonated, or copolymers of methacrylic acid and divinylbenzene, including those available commercially as Dowex® resins or Amberlite® resins. The resin may be in acid form or in the form of a salt with an alkali metal, (e.g., sodium or potassium). Particularly preferred is Amberlite® which is an insoluble, weakly acidic, hydrogen form, cation exchange resin supplied as dry, fine powder. It is derived from a porous copolymer of methacrylic acid and divinylbenzene.

The “taste-masked dispersible tablet”, as recited herein, means that the tablet is to be dispersed in a sufficient quantity of water to form a taste-masked dispersion, prior to administration. The underlying principle of this particular tablet is that the said drug and the said cation exchange resin are present in an un-complexed form in the tablet. Upon dispersing the tablet in water prior to administration, the said drug and the said cation exchange resin form a complex within 30 seconds to 15 minutes of dispersing the said tablet in water. This results in the desired taste-masking of the drug.

The taste-masked dispersible tablets may further comprise one or more of other pharmaceutically acceptable excipients that are routinely used and may be selected from diluents, binders, lubricants/glidants, disintegrants, flavoring agents, sweetening agents, coloring agents, or combinations thereof.

Suitable diluents that may be used include, but are not limited to, microcrystalline cellulose, silicified microcrystalline cellulose, microfine cellulose, lactose, starch, pregelatinized starch, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, or combinations thereof.

Suitable binders that may be used include, but are not limited to, acacia, guar gum, alginic acid, carbomer, dextrin, maltodextrin, methylcellulose, ethylcellulose, hydroxyethylcellulose, hydroxypropylcellulose, hydroxypropylmethylcellulose, carboxymethylcellulose sodium, magnesium aluminum silicate, polymethacrylates, crospovidones, povidones, copovidones, gelatin, starch, or combinations thereof. When a wet granulation process is followed for the preparation of the tablets, the granulating fluid can be either a solvent or a binder dissolved in a solvent. The solvents that may be used include, but are not limited to, dichloromethane, ethyl alcohol, or isopropyl alcohol.

Suitable lubricants/glidants that that may be used include, but are not limited to, magnesium stearate, zinc stearate, calcium stearate, stearic acid, colloidal silicon dioxide, glyceryl palmitostearate, vegetable oils, polyethylene glycols, polyvinyl alcohols, talc, sodium benzoate, sodium stearyl fumarate, magnesium oxide, poloxamer, sodium lauryl sulphate, polyoxyethylene monostearate, cocoa butter, hydrogenated vegetable oils, mineral oil, polysaccharides, or combinations thereof.

Suitable disintegrants that may be used include, but are not limited to, cross linked calcium or sodium carboxy methyl cellulose, starch, sodium starch glycolate, pregelatinized starch, crosslinked polyvinyl pyrrolidone, low-substituted hydroxypropyl cellulose, microcrystalline cellulose, ion exchange resin, cross-linked polyacrylic acid, alginates, colloidal magnesium-aluminum silicate, calcium silicate, or combinations thereof.

Additional taste-masking agents that may be used include flavoring agents and sweetening agents. Flavoring agents may be chosen from natural and synthetic flavor liquids and include, but are not limited to, volatile oils, synthetic flavor oils, flavoring aromatics, oils, liquids, oleoresins or extracts derived from plants, leaves, flowers, fruits, stems, or combinations thereof. The sweetening agents may be chosen from the following non-limiting list: sucrose, dextrose, invert sugar, fructose, and mixtures thereof; saccharin, aspartame, acesulfame, sucralose; or sugar alcohols such as sorbitol, mannitol and xylitol.

Suitable coloring agents that may be used include, but are not limited to, titanium dioxide pigments, lake colors, and iron oxide pigments.

The taste-masked dispersible tablets, as defined herein, may be manufactured using conventional tabletting techniques like direct compression, dry granulation, or wet granulation.

Preferred taste-masked dispersible tablets of the present invention may take the form of several different embodiments.

In one embodiment, the present invention relates to a taste-masked dispersible tablet comprising a drug, the cation exchange resin Amberlite® IRP64, and other pharmaceutically acceptable excipients.

In another embodiment, the drug is tenofovir disoproxil fumarate, the cation exchange resin is Amberlite® IRP64, and the other pharmaceutically acceptable excipients are sucralose, flavor, croscarmellose sodium, mannitol, magnesium stearate, and colloidal silicon dioxide.

In another embodiment, the drug is tenofovir disoproxil fumarate, the cation exchange resin is Amberlite® IRP64, and the other pharmaceutically acceptable excipients are microcrystalline cellulose, sucralose, flavor, corscarmellose sodium, ethylcellulose, mannitol, xylitol, and magnesium stearate.

In another embodiment, the drug is tenofovir disoproxil fumarate, the cation exchange resin is Amberlite® IRP64, and the other pharmaceutically acceptable excipients are microcrystalline cellulose, sucralose, flavor, croscarmellose sodium, ethylcellulose, crospovidone, mannitol, xylitol, and magnesium stearate.

In another embodiment, the present invention relates to a process for the preparation of a taste-masked dispersible tablet comprising tenofovir disoproxil fumarate, Amberlite® IRP64, and other pharmaceutically acceptable excipients, wherein the process comprises the steps of:
a) sifting the ingredients, i.e., tenofovir disoproxil fumarate, Amberlite® IRP64, and other pharmaceutically acceptable excipients separately through suitable sieves;
b) blending the sifted ingredients in a blender for a suitable time;
c) compressing the resultant blend into a dispersible tablet using appropriate tooling.

In another embodiment, the present invention relates to a process for the preparation of a taste-masked dispersible tablet comprising tenofovir disoproxil fumarate, Amberlite® IRP64, and other pharmaceutically acceptable excipients, wherein the process comprises the steps of:
a) sifting the ingredients, i.e., tenofovir disoproxil fumarate, Amberlite® IRP64, and other pharmaceutically acceptable excipients through suitable sieves;
b) blending the sifted ingredients in a blender for a suitable time;
c) granulating the resultant blend with a binder solution;
d) drying the granules;
e) mixing the granules with the extragranular excipient(s); and
f) compressing the resultant blend into a dispersible tablet using appropriate tooling.

In another embodiment, the present invention relates to a method of orally administering to a human, a taste-masked dispersible tablet as described in the above embodiments, wherein the method comprises dispersing the tablet in a sufficient quantity of water prior to administration so that tenofovir disoproxil fumarate and Amberlite® IRP64 form a complex within 30 seconds to 15 minutes of dispersing the said taste-masked dispersible tablet in water. The sufficient quantity of water may be from 1 mL to 200 mL or a quantity of water comfortably consumed by the human, such as a glass of water routinely consumed.

From the above, it is apparent that various modifications and combinations of the dispersible tablets detailed in the text may be made without departing from the spirit and scope of the invention. The invention, as described herein, may be illustrated by the following examples but is not to be construed to be limited by them.

### EXAMPLES

### Examples 1-4

| | | Quantity (mg/tablet) | | | |
|---|---|---|---|---|---|
| | | | | | Example |
| S. No. | Ingredients | Example 1 | Example 2 | Example 3 | 4 |
| 1. | Tenofovir disoproxil | 70.0 | 150.0 | 100.0 | 100.0 |
| | fumarate | | | | |
| 2. | Amberlite ® IRP64 | 210.0 | 450.0 | 45.0 | 45.0 |
| 3. | Povidone | — | — | 3.0 | 10.0 |
| 4. | Microcrystalline | — | — | 100.0 | 205.0 |
| | cellulose | | | | |
| 5. | Ethylcellulose | — | — | 7.0 | 14.0 |
| 6. | Hydroxypropyl | — | — | 50.0 | |
| | cellulose | | | | |
| 7. | Sucralose | 70.0 | 150.0 | 75.0 | 75.0 |
| 8. | Flavor | 15.0 | 15.0 | 20.0 | 15.0 |
| 9. | Croscarmellose | 20.0 | 20.0 | 60.0 | 60.0 |
| | sodium | | | | |
| 10. | Crospovidone | — | — | — | 25.0 |
| 11. | Mannitol | 109.0 | 110.0 | 305.0 | 266.0 |
| 12. | Xylitol | — | — | 125.0 | 125.0 |
| 13. | Magnesium stearate | 2.0 | 2.5 | 10.0 | 10.0 |
| 14. | Colloidal silicon | 4.0 | 4.5 | — | — |
| | dioxide | | | | |
| 15. | Dichloromethane | — | — | q.s.* | q.s.* |
| | Total Weight | 500.0 | 902.0 | 900.0 | 950.0 |

### Procedures:

### Examples 1-2

a) All the above ingredients were sifted separately through suitable sieves;
b) The sifted ingredients were blended in a blender for a suitable time; and
c) The resultant blend was compressed into dispersible tablet using appropriate tooling.

### Examples 3-4

a) Tenofovir disoproxil fumarate, microcrystalline cellulose (first portion; Example 4), and Amberlite® IRP64 were sifted through a suitable mesh;
b) The sifted ingredients of step a) were transferred into a rapid mixer granulator and dry mixed for a suitable time;
c) Ethylcellulose and povidone were dissolved in dichloromethane, and the solution thus formed was used to granulate the mixture of step b) in the rapid mixer granulator;
d) The resultant granules were dried in a fluid bed dryer and sifted through a suitable mesh to get uniform size granules;
e) Mannitol, sucralose, xylitol, croscarmellose sodium, microcrystalline cellulose (second portion; Example 4), and crospovidone (Example 4) were sifted through a suitable mesh;
f) The sifted extragranular ingredients of step e) were mixed with the dried granules of step d) in a suitable blender;
g) Flavor and magnesium stearate were sifted through a suitable mesh and mixed with the blend of step f); and
h) The resultant blend was compressed into dispersible tablets using appropriate tooling.

A taste-masked dispersible tablet comprising a drug-resin complex was prepared as per the usual complexation method for comparison and is described in Table 1 below:

TABLE 1
Comparative Example of a taste-masked dispersible tablet comprising the
drug resin-complex prepared as per the usual complexation method

| Ingredients | Quantity in mg/tablet |
|---|---|
| Tenofovir + Amberlite ® IRP64 resin complex | 280.00 |
| Polyplasdone ® XL | 20.00 |
| Aspartame | 15.00 |
| Acesulfame Potassium | 7.00 |
| Lactose | 22.00 |
| Magnesium stearate | 3.00 |
| Mint flavor | 3.00 |
| Total weight | 350.00 |

### Procedure for the Comparative Example:

### (I) Drug-Resin Complex Preparation Process

a) A required quantity of Amberlite IRP64 was added to distilled water to make about 10% w/w slurry;
b) The slurry was stirred for a sufficient time to get a uniform/lump free dispersion;
c) A specified quantity of tenofovir disoproxil fumarate was added slowly under stirring to the above dispersion to make a drug:resin ratio of 1:3;
d) The entire mixture was stirred for about 4 hours and the dispersion was kept aside for setting;
e) The solid resin was separated by filtration and subsequently dried to get the desired tenofovir disoproxil fumarate-Amberlite® IRP64 complex.

### (II) Tablet Preparation Process

a) The drug-resin complex prepared as per the above process, magnesium stearate, and all other excipients were separately sifted through suitable sieves;
b) The above sifted ingredients were mixed for a sufficient time to get a uniform blend;
c) The resultant blend was compressed into a dispersible tablet using appropriate tooling.

A comparative stability analysis was performed by subjecting the tablets prepared as per the above Example 1 and tablets prepared as per the Comparative Example to an accelerated stability testing at 40° C./75% relative humidity. The assay values are summarized in Table 2 below.

TABLE 2
Assay results of tenofovir disoproxil fumarate in taste-masked dispersible
tablets prepared as per the above Example 1 and the Comparative
Example when stored at 40° C./75% relative humidity

| | | Assay Results | |
|---|---|---|---|
| S. No. | Stability Condition | Example 1 | Comparative Example |
| 1. | Initial | 94.0% | 89.3% |
| 2. | 1 month 40° C./75% RH | 94.0% | 78.6% |
| 3. | 2 month 40° C./75% RH | 95.4% | Not performed |
| 4. | 3 month 40° C./75% RH | 94.5% | Not performed |

The assay results clearly indicate that the tablets in which the drug-resin complex is formed in situ exhibit better stability than the tablets comprising the drug-resin complex prepared by the usual complexation method.

We claim:

1. A taste-masked dispersible tablet comprising a drug, a cation exchange resin, and other pharmaceutically acceptable excipients, such that the said drug and the said cation exchange resin are present in an un-complexed form in the tablet.

2. The taste-masked dispersible tablet according to claim 1, wherein the said drug and the said cation exchange resin form a complex within 30 seconds to 15 minutes of dispersing the taste-masked dispersible tablet in water.

3. The taste-masked dispersible tablet according to claim 1, wherein the cation exchange resin is Amberlite® IRP64.

4. The taste-masked dispersible tablet according to claim 1, wherein the other pharmaceutically acceptable excipients are selected from diluents, binders, lubricants/glidants, disintegrants, flavoring agents, sweetening agents, coloring agents, and combinations thereof.

5. A process for the preparation of a taste-masked dispersible tablet according to claim 1, wherein the process comprises the steps of direct compression, dry granulation, or wet granulation.

6. A method of orally administering to a human a taste-masked dispersible tablet comprising a drug, a cation exchange resin, and other pharmaceutically acceptable excipients, such that the said drug and the said cation exchange resin are present in an un-complexed form in the tablet, wherein the method comprises dispersing the said tablet in a sufficient quantity of water prior to administration.

7. A taste-masked dispersible tablet comprising tenofovir disoproxil fumarate, a cation exchange resin, and other pharmaceutically acceptable excipients, such that tenofovir disoproxil fumarate and the cation exchange resin are present in an un-complexed form in the tablet.

8. The taste-masked dispersible tablet according to claim 7, wherein the cation exchange resin is Amberlite® IRP64.
